(19) United States
(12) Patent Application Publication
(10) Pub. No.: US 2023/0404907 A1
(43) Pub. Date: Dec. 21, 2023

(54) Monolithic Implantable Device for Sustained Release of an Antibody

(71) Applicant: Celanese EVA Performance Polymers LLC, Irving, TX (US)

(72) Inventors: Jeffrey C. Haley, Norwood, OH (US); Vijay Gyanani, Powell, OH (US); Harsh Patel, Union, KY (US)

(21) Appl. No.: 18/332,826

(22) Filed: Jun. 12, 2023

Related U.S. Application Data
(60) Provisional application No. 63/358,345, filed on Jul. 5, 2022.
(60) Provisional application No. 63/351,471, filed on Jun. 13, 2022.

(51) Int. Cl. A61K 9/00; A61K 47/69; C07K 16/22; A61K 47/26

(52) U.S. Cl. CPC A61K 9/0004; A61K 47/26; C07K 16/22; A61K 47/6935

(57) ABSTRACT

A monolithic implantable device for delivery of an antibody is provided. The implantable device comprises a polymer matrix within which is dispersed a pharmaceutical formulation that includes one or more therapeutic agents and optionally, one or more excipients. The therapeutic agents contain an antibody and the polymer matrix contains a hydrophobic polymer. Within a time period of 35 days, the device exhibits a cumulative weight-based release ratio of the antibody of from about 20% to about 60%.

### RELATED APPLICATIONS

The present application is based upon and claims priority to U.S. Provisional Patent Application Ser. No. 63/351,471, having a filing date of Jun. 13, 2022, and U.S. Provisional Patent Application Ser. No. 63/358,345, having a filing date of Jul. 5, 2022, which are incorporated herein by reference.

### BACKGROUND OF THE INVENTION

Antibodies are typically composed of one or more oligomeric or polymeric chains, forming a three-dimensional structure held together by non-covalent forces. While antibodies have the potential for a multitude of therapeutic benefits, it has been traditionally difficult to controllably deliver these compounds over a sustained period of time. Many implantable delivery devices, for example, are formed by dispersing a pharmaceutical formulation into a matrix polymer. These dispersed drug molecules can diffuse through the implant and be released into a patient. Unfortunately, however, drug elution is highly dependent upon the diffusion coefficient of the drug molecule, which in turn, generally decreases with increasing molecular weight of the drug molecule. Thus, antibodies tend to have a lower diffusion coefficient due to their larger molecular weight. Further, such compounds often have chain length entanglements, which can even further reduce the effective diffusion coefficient. Various attempts have been made to help improve and control the release rate of certain types of drug compounds from an implantable device. U.S. Patent Publication No. 2021/0007973, for example, describes a device that contains an outer shell that is applied to an inner implant and that contains a mixture of a polymer matrix and a hydrophilic porogen (e.g., ethylcellulose). Unfortunately, the use of such porogenic shells is not always effective for the release of antibodies and, in any event, the additional shell can make the device overly complicated and costly to manufacture.

In light of these difficulties, a need continues to exist for an implantable delivery device that is capable of delivering an antibody in effective amounts over a sustained period of time.

### SUMMARY OF THE INVENTION

In accordance with one embodiment of the present invention, a monolithic implantable device for delivery of an antibody is disclosed. The implantable device comprises a polymer matrix within which is dispersed a pharmaceutical formulation that includes one or more therapeutic agents and optionally, one or more excipients. The therapeutic agents contain an antibody and the polymer matrix contains a hydrophobic polymer. Within a time period of 35 days, the device exhibits a cumulative weight-based release ratio of the antibody of from about 20% to about 60%.

Other features and aspects of the present invention are set forth in greater detail below.

### BRIEF DESCRIPTION OF THE FIGURES

A full and enabling disclosure of the present invention, including the best mode thereof, directed to one of ordinary skill in the art, is set forth more particularly in the remainder of the specification, which makes reference to the appended figures in which:

FIG. 1 is a graph showing the cumulative weight-based release percentage of an IgG antibody pharmaceutical formulation versus release time (hours) for Examples 1-3;

FIG. 2 is a graph showing the cumulative release of the surface area versus time for Examples 1-3;

FIG. 3 is a graph showing the cumulative weight-based release percentage of a trastuzumab pharmaceutical formulation versus release time (hours) for Example 4;

FIG. 4 is a graph showing the cumulative weight-based release percentage of lysozyme versus release time (hours) for Examples 5-6;

FIG. 5 is an SEC chromatogram for Example 7;

FIG. 6 is an SEC chromatogram for Example 8;

FIG. 7 is an SEC chromatogram for Example 9;

FIG. 8 is an SEC chromatogram for Example 10; and

FIG. 9 is an SEC chromatogram for Example 11.

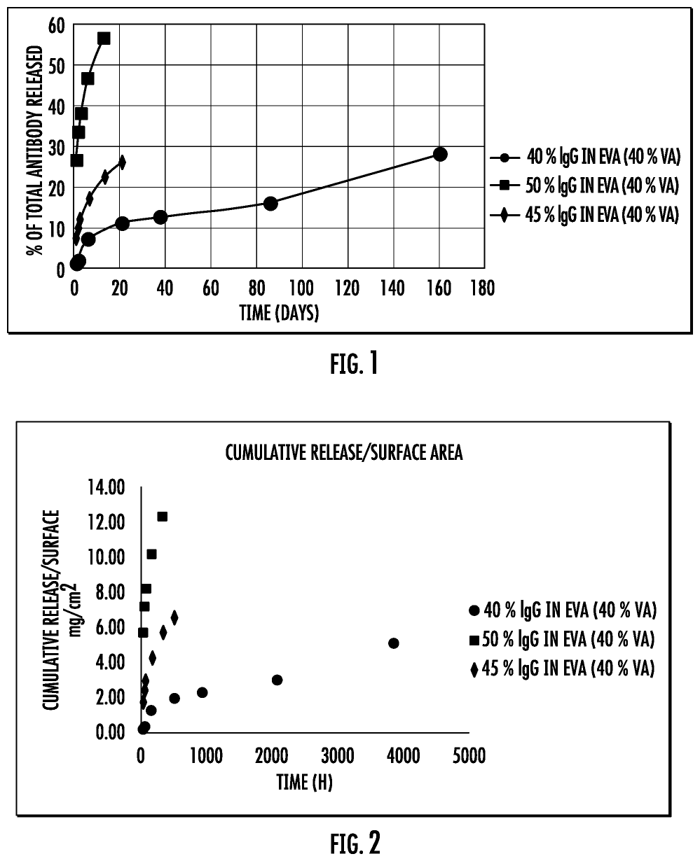

Repeat use of references characters in the present specification and drawing is intended to represent same or analogous features or elements of the invention.

### DETAILED DESCRIPTION

It is to be understood by one of ordinary skill in the art that the present discussion is a description of exemplary embodiments only, and is not intended as limiting the broader aspects of the present invention.

Generally speaking, the present invention is directed to a monolithic implantable device that is capable of delivering an antibody that can potentially prohibit and/or treat a condition, disease, and/or cosmetic state in a patient (e.g., human, pet, farm animal, etc.). As used herein, the term “monolithic” generally means that the device is formed from a single constituent layer or member, which is often referred to as a “core.” Thus, the device generally lacks additional release layers, such as shells, membranes, sheaths, etc., such that the core itself can define an exterior peripheral surface of the device. The monolithic implantable device contains a polymer matrix that includes a hydrophobic polymer and a pharmaceutical formulation that is dispersed within the polymer matrix. The pharmaceutical formulation includes one or more therapeutic agents that include the antibody and one or more optional excipients, such as buffering agents, stabilizers (e.g., surfactants, saccharides, etc.), and so forth.

The weight ratio of therapeutic agents (e.g., antibody) to the polymer matrix may vary depending on the presence of any optional excipients. For example, when only therapeutic agents are present, the weight ratio of the therapeutic agents to the polymer matrix is typically controlled within a range of from about 0.7 to about 1, in some embodiments from about 0.75 to about 1, in some embodiments from about 0.75 to about 0.9, and in some embodiments, from about 0.8 to about 0.85. On the other hand, when excipients are present, the weight ratio of the therapeutic agents to the polymer matrix may be from about 0.3 to about 1, in some embodiments from about 0.75 to about 1, in some embodiments from about 0.35 to about 0.5, and in some embodiments, from about 0.38 to about 0.45. Regardless, the present inventors have surprisingly discovered that the cumulative release rate of from the device can be controlled within a desired range when the weight ratio of the overall pharmaceutical formulation to the polymer matrix is kept within a range of from about 0.7 to about 1, in some embodiments from about 0.75 to about 1, in some embodiments from about 0.75 to about 0.9, and in some embodiments, from about 0.8 to about 0.85. For example, the pharmaceutical formulation may constitute from about 30 wt. % to about 50 wt. %, in some embodiments from about 35 wt. % to about 50 wt. %, in some embodiments from about 40 wt. % to about 50 wt. %, and in some embodiments, from about 42 wt. % to about 48 wt. % of the device. Likewise, the polymer matrix may constitute from about 50 wt. % to about 70 wt. %, in some embodiments from about 50 wt. % to about 65 wt. %, in some embodiments from about 50 wt. % to about 60 wt. %, and in some embodiments, from about 52 wt. % to about 58 wt. % of the device.

Through selective control over the particular nature of the specific nature and concentration of the components of the implantable device, the present inventors have discovered that the resulting device can be effective for sustained release over an antibody over a prolonged period of time. For example, the implantable device can release an antibody for a time period of about 5 days or more, in some embodiments about 10 days or more, in some embodiments from about 20 days to about 210 days, and in some embodiments, from about 30 days to about 180 days. Further, the present inventors have also discovered that an antibody can be released in a highly controlled manner over the course of the release time period. After a time period of 15 days, for example, the cumulative weight-based release ratio of an antibody may be from about 10% to about 55%, in some embodiments from about 12% to about 40%, and in some embodiments, from about 15% to about 30%. Likewise, after a time period of 35 days, the cumulative weight-based release ratio of an antibody may be from about 20% to about 60%, in some embodiments from about 30% to about 55%, and in some embodiments, from about 35% to about 50%. The “cumulative weight-based release ratio” may be determined by dividing the total amount of an antibody released at a particulate time interval by the total amount of the antibody initially present, and then multiplying this number by 100. Furthermore, after a time period of 35 days, the cumulative surface area-based release ratio may be from about 5 to about 70 mg/cm², in some embodiments from about 10 to about 50 mg/cm², and in some embodiments, from about 15 to about 40 mg/cm². Likewise, after a time period of 90 days, the cumulative surface area-based release ratio may be from about 15 to about 70 mg/cm², in some embodiments from about 20 to about 60 mg/cm², and in some embodiments, from about 30 to about 50 mg/cm². Furthermore, after a time period of 120 days, the cumulative surface area-based release ratio may be from about 30 to about 70 mg/cm², in some embodiments from about 35 to about 65 mg/cm², and in some embodiments, from about 40 to about 50 mg/cm². The “cumulative surface-based release ratio” may be determined by dividing the amount of an antibody released at a particulate time interval (“mg”) by the surface area of the implantable device from which the antibody can be released (“cm²”).

Various embodiments of the present invention will now be described in more detail.

### I. Polymer Matrix

As indicated above, the polymer matrix includes at least one polymer that is generally hydrophobic in nature so that it can retain its structural integrity for a certain period of time when placed in an aqueous environment, such as the body of a mammal, and stable enough to be stored for an extended period before use. Examples of suitable hydrophobic polymers for this purpose may include, for instance, silicone polymer, polyolefins, polyvinyl chloride, polycarbonates, polysulphones, styrene acrylonitrile copolymers, polyurethanes, silicone polyether-urethanes, polycarbonate-urethanes, silicone polycarbonate-urethanes, etc., as well as combinations thereof. Of course, hydrophilic polymers that are coated or otherwise encapsulated with a hydrophobic polymer are also considered “hydrophobic polymers” and suitable for use in the polymer matrix. The melt flow index of the hydrophobic polymer(s) and resulting polymer matrix may also range from about 0.2 to about 100 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-20 at a temperature of 190° C. and a load of 2.16 kilograms. The density of the hydrophobic polymer(s) and resulting polymer matrix may also range from about 0.900 to about 1.00 gram per cubic centimeter (g/cm³), in some embodiments from about 0.910 to about 0.980 g/cm³, and in some embodiments, from about 0.940 to about 0.970 g/cm³, as determined in accordance with ASTM D1505-18. Further, the melting temperature of the hydrophobic polymer(s) and resulting polymer matrix may likewise range from about 40° C. to about 140° C., in some embodiments from about 50° C. to about 125° C., and in some embodiments, from about 60° C. to about 120° C., as determined in accordance with ASTM D3418-21.

In certain embodiments, the polymer matrix may contain a semi-crystalline olefin copolymer. Such copolymers are generally derived from at least one olefin monomer (e.g., ethylene, propylene, etc.) and at least one polar monomer that is grafted onto the polymer backbone and/or incorporated as a constituent of the polymer (e.g., block or random copolymers). Suitable polar monomers include, for instance, a vinyl acetate, vinyl alcohol, maleic anhydride, maleic acid, (meth)acrylic acid (e.g., acrylic acid, methacrylic acid, etc.), (meth)acrylate (e.g., acrylate, methacrylate, ethyl acrylate, methyl methacrylate, ethyl methacrylate, etc.), and so forth. A wide variety of such copolymers may generally be employed in the polymer composition, such as ethylene vinyl acetate copolymers, ethylene (meth)acrylic acid polymers (e.g., ethylene acrylic acid copolymers and partially neutralized ionomers of these copolymers, ethylene methacrylic acid copolymers and partially neutralized ionomers of these copolymers, etc.), ethylene (meth)acrylate polymers (e.g., ethylene methylacrylate copolymers, ethylene ethyl acrylate copolymers, ethylene butyl acrylate copolymers, etc.), and so forth. Regardless of the particular monomers selected, certain aspects of the copolymer can be selectively controlled to help achieve the desired release properties. For instance, the polar monomer content of the copolymer may be selectively controlled to be within a range of from about 10 wt. % to about 60 wt. %, in some embodiments from about 20 wt. % to about 60 wt. %, in some embodiments from about 25 wt. % to about 50 wt. %, in some embodiments from about 30 wt. % to about 48 wt. %, and in some embodiments, from about 35 wt. % to about 45 wt. % of the copolymer. Conversely, the olefin monomer content of the copolymer may likewise be within a range of from about 40 wt. % to about 90 wt. %, in some embodiments from about 40 wt. % to about 80 wt. %, in some embodiments from about 50 wt. % to about 75 wt. %, in some embodiments from about 50 wt. % to about 80 wt. %, in some embodiments from about 52 wt. % to about 70 wt. %, and in some embodiments, from about 55 wt. % to about 65 wt. %. Among other things, such a comonomer content can help achieve a controllable, sustained release profile of the pharmaceutical formulation, while also still having a relatively low melting temperature that is more similar in nature to the melting temperature of the hydrophobic polymer(s).

In one particular embodiment, for example, the polymer matrix may contain at least one ethylene vinyl acetate polymer, which is a copolymer that is derived from at least one ethylene monomer (olefin monomer) and at least one vinyl acetate monomer (polar monomer). The melting temperature, monomer content, melt flow index, and/or density may be within the ranges noted above. Particularly suitable examples of ethylene vinyl acetate copolymers that may be employed include those available from Celanese under the designation ATEVA® (e.g., ATEVA® 4030AC); Dow under the designation ELVAX® (e.g., ELVAX® 40W); and Arkema under the designation EVATANE® (e.g., EVATANE 40-55).

Any of a variety of techniques may generally be used to form the ethylene vinyl acetate copolymer(s) with the desired properties as is known in the art. In one embodiment, the polymer is produced by copolymerizing an ethylene monomer and a vinyl acetate monomer in a high pressure reaction. Vinyl acetate may be produced from the oxidation of butane to yield acetic anhydride and acetaldehyde, which can react together to form ethylidene diacetate. Ethylidene diacetate can then be thermally decomposed in the presence of an acid catalyst to form the vinyl acetate monomer. Examples of suitable acid catalysts include aromatic sulfonic acids (e.g., benzene sulfonic acid, toluene sulfonic acid, ethylbenzene sulfonic acid, xylene sulfonic acid, and naphthalene sulfonic acid), sulfuric acid, and alkanesulfonic acids, such as described in U.S. Pat. No. 2,425,389 to Oxley et al.; U.S. Pat. No. 2,859,241 to Schnizer; and U.S. Pat. No. 4,843,170 to Isshiki et al. The vinyl acetate monomer can also be produced by reacting acetic anhydride with hydrogen in the presence of a catalyst instead of acetaldehyde. This process converts vinyl acetate directly from acetic anhydride and hydrogen without the need to produce ethylidene diacetate. In yet another embodiment, the vinyl acetate monomer can be produced from the reaction of acetaldehyde and a ketene in the presence of a suitable solid catalyst, such as a perfluorosulfonic acid resin or zeolite.

In certain embodiments, it may also be desirable to employ blends of a first hydrophobic polymer and a second hydrophobic polymer such that the overall blend and polymer matrix have a melting temperature and/or melt flow index within the range noted above. For example, the polymer matrix may contain a first hydrophobic polymer (e.g., ethylene vinyl acetate copolymer) and a second hydrophobic polymer (e.g., ethylene vinyl acetate copolymer) having a melting temperature that is greater than the melting temperature of the first polymer. The second polymer may likewise have a melt flow index that is the same, lower, or higher than the corresponding melt flow index of the first polymer. The first polymer may, for instance, have a melting temperature of from about 20° C. to about 60° C., in some embodiments from about 25° C. to about 55° C., and in some embodiments, from about 30° C. to about 50° C., such as determined in accordance with ASTM D3418-21, and/or a melt flow index of from about 40 to about 900 g/10 min, in some embodiments from about 50 to about 500 g/10 min, and in some embodiments, from about 55 to about 250 g/10 min, as determined in accordance with ASTM D1238-20 at a temperature of 190° C. and a load of 2.16 kilograms. The second polymer may likewise have a melting temperature of from about 50° C. to about 100° C., in some embodiments from about 55° C. to about 90° C., and in some embodiments, from about 60° C. to about 80° C., such as determined in accordance with ASTM D3418-21, and/or a melt flow index of from about 0.2 to about 55 g/10 min, in some embodiments from about 0.5 to about 50 g/10 min, and in some embodiments, from about 1 to about 40 g/10 min, as determined in accordance with ASTM D1238-20 at a temperature of 190° C. and a load of 2.16 kilograms. The first polymer may constitute from about 20 wt. % to about 80 wt. %, in some embodiments from about 30 wt. % to about 70 wt. %, and in some embodiments, from about 40 wt. % to about 60 wt. % of the polymer matrix, and the second polymer may likewise constitute from about 20 wt. % to about 80 wt. %, in some embodiments from about 30 wt. % to about 70 wt. %, and in some embodiments, from about 40 wt. % to about 60 wt. % of the polymer matrix.

Typically, hydrophobic polymer(s), such as ethylene vinyl acetate copolymer(s), constitute the entire polymer matrix. In other words, the polymer matrix may be generally free of hydrophilic polymers, such as alkyl celluloses and hydroxyalkyl celluloses (e.g., ethylcellulose, methylcellulose, and hydroxymethylcellulose), polyvinylpyrrolidone, and so forth. Namely, hydrophilic polymers generally constitute no more than about 10 wt. % of the polymer matrix, in some embodiments no more than about 5 wt. % of the polymer matrix, and in some embodiments, from 0 wt. % to about 2 wt. % of the polymer matrix (e.g., 0 wt. %). For example, ethylene vinyl acetate copolymers may constitute the entire polymer matrix. In other embodiments, ethylene vinyl acetate copolymers may be blended with other types of hydrophobic polymers. In such embodiments, ethylene vinyl acetate copolymer(s) may constitute from about from about 70 wt. % to about 99.999 wt. %, in some embodiments from about 80 wt. % to about 99.99 wt. %, and in some embodiments, from about 90 wt. % to about 99.9 wt. % of the polymer content of the polymer matrix, while other hydrophobic polymers constitute from about 0.001 wt. % to about 30 wt. %, in some embodiments from about 0.01 wt. % to about 20 wt. %, and in some embodiments, from about 0.1 wt. % to about 10 wt. % of the polymer content of the polymer matrix.

### II. Pharmaceutical Formulation

A. Therapeutic Agents

A pharmaceutical formulation is also dispersed within the polymer matrix that contains one or more therapeutic agents, which include at least one antibody that can be capable of prohibiting and/or treating a condition, disease, and/or cosmetic state a patient. As used herein, the term “antibody” generally includes, by way of example, both naturally occurring and non-naturally occurring Abs, monoclonal and polyclonal Abs, chimeric and humanized Abs; human or nonhuman Abs, wholly synthetic Abs, single chain Abs, etc. A nonhuman Ab may be humanized by recombinant methods to reduce its immunogenicity in man. The term “antibody” also includes an antigen-binding fragment or an antigen-binding portion of any of the aforementioned immunoglobulins, and includes a monovalent and a divalent fragment or portion, and a single chain Ab. Particularly suitable antibodies may include monoclonal antibodies (“MAbs”), multispecific (e.g., bispecific) antibodies, or combinations thereof. The term “monoclonal antibody” generally refers to a non-naturally occurring preparation of Ab molecules of single molecular composition, i.e., Ab molecules whose primary sequences are essentially identical, and which exhibits a single binding specificity and affinity for a particular epitope. Multispecific antibodies, on the other hand, can bind simultaneously different antigens (e.g., two antigens). Such antibodies are generally produced by hybridoma, recombinant, transgenic or other techniques known to those skilled in the art. A “human” antibody refers to an Ab having variable regions in which both the framework and CDR regions are derived from human germline immunoglobulin sequences. Furthermore, if the Ab contains a constant region, the constant region also is derived from human germline immunoglobulin sequences. The human Abs may include amino acid residues not encoded by human germline immunoglobulin sequences (e.g., mutations introduced by random or site-specific mutagenesis in vitro or by somatic mutation in vivo). However, the term “human antibody”, as used herein, is not intended to include Abs in which CDR sequences derived from the germline of another mammalian species, such as a mouse, have been grafted onto human framework sequences.

In some embodiments, the antibody (includes a fragment thereof) can neutralize, block, inhibit, abrogate, reduce, and/or interfere with one or more biological activities (e.g., mitogenic, angiogenic and/or vascular permeability) of a proliferating cell. Such antibodies may, for instance, bind to HER2, TNF-α, VEGF-A, α4-integrin, CD20, CD52, CD25, CD11a, EGFR, respiratory syncytial virus (RSV), glycoprotein IIb/IIIa, IgG1, IgE, complement component 5 (C5), B-cell activating factor (BAFF), CD19, CD30, interleukin-1 beta (IL1β), prostate specific membrane antigen (PSMA), CD38, RANKL, GD2, SLAMF7 (CD319), proprotein convertase subtilisin/kexin type 9 (PCSK9), dabigatran, cytotoxic T-lymphocyte-associated protein 4 (CTLA4), interleukin-5 (IL-5), programmed cell death protein (PD-1), VEGFR2 (KDR), protective antigen (PA) of B. anthracis, interleukin-17 (IL-17), interleukin-6 (IL-6), interleukin-6 receptor (IL6R), interleukin-12 (IL-12), interleukin 23 (IL-23), sclerostin (SOST), myostatin (GDF-8), activin receptor-like kinase 1, delta like ligand 4 (DLL4), angiopoietin 3, VEGFR1, selectin, oxidized low-density lipoprotein (oxLDL), platelet-derived growth factor receptor beta, neuropilin 1, Von Willebrand factor (vWF), neural apoptosis-regulated proteinase 1, beta-amyloid, reticulon 4 (RTN4)/Neurite Outgrowth Inhibitor (NOGO-A), nerve growth factor (NGF), LINGO-1, myelin-associated glycoprotein, etc., as well as combinations thereof.

In one particular embodiment, for example, the antibody may be an anti-PD-1 and/or anti-PD-L1 antibody, such as employed as immune checkpoint inhibitors for treating cancer. PD-1 (or Programmed Death-1) refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863. PD-L1 (or Programmed Death Ligand-1) is one of two cell surface glycoprotein ligands for PD-1 (the other being PD-L2) that downregulate T cell activation and cytokine secretion upon binding to PD-1. The term “PD-L1” as used herein includes human PD-L1 (hPD-LI), variants, isoforms, and species homologs of hPD-LI, and analogs having at least one common epitope with hPD-LI. The complete hPD-LI sequence can be found under GenBank Accession No. Q9NZQ7. HuMAbs that bind specifically to PD-1 with high affinity have been described, for instance, in U.S. Pat. Nos. 8,008,449 and 8,779,105. Other anti-PD-1 mAbs have been described in, for example, U.S. Pat. Nos. 6,808,710, 7,488,802, 8,168,757 and 8,354,509, and PCT Publication No. WO 2012/145493. For example, the anti-PD-1 MAb may be nivolumab. Nivolumab (also known as Opdivo®; formerly designated 5C4, BMS-936558, MDX-1106, or ONO-4538) is a fully human IgG4 (S228P) PD-1 immune checkpoint inhibitor Ab that selectively prevents interaction with PD-1 ligands (PD-LI and PD-L2), thereby blocking the down-regulation of antitumor T-cell functions (U.S. Pat. No. 8,008,449). In another embodiment, the anti-PD-1 mAb is pembrolizumab, as well as antigen-binding variants thereof. Pembrolizumab (also known as Keytruda®, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. Nos. 8,354,509 and 8,900,587). In other embodiments, the anti-PD-1 MAb is MED10608 (formerly AMP-514) as described, for example, in U.S. Pat. No. 8,609,089. Yet other examples of humanized monoclonal antibodies include Pidilizumab (CT-011), BGB-A317, etc., as well as antigen-binding variants thereof.

Another example of a suitable antibody is an anti-VEGF antibody, which is an antibody or antibody fragment (e.g., Fab or a scFV fragment) that specifically binds to a VEGF receptor. Anti-VEGF antibodies act, for example, by interfering with the binding of VEGF to a cellular receptor, by interfering with vascular endothelial cell activation after VEGF binding to a cellular receptor, and/or by killing cells activated by VEGF. An anti-VEGF antibody will usually not bind to other VEGF homologues (e.g., VEGF-B or VEGF-C) or other growth factors (e.g., PIGF, PDGF or bFGF). Suitable anti-VEGF antibodies may include monoclonal and/or bispecific anti-VEGF antibodies, such as A4.6.1, bevacizumab, ranibizumab, G6, B20, 2C3, and other antibodies such as described in U.S. Pat. Nos. 6,582,959, 6,703,020, 7,060,269, 7,169,901, 7,691,977, and 10,590,193; U.S. Patent Publication No. 2009/0169556; WO 94/10202; WO 98/45332; WO 96/30046, WO 2019/154776; WO 2010/040508; WO 2011/117329; WO 2012/131078; WO 2015/083978; WO 2017/197199; and WO 2014/009465, all of which are incorporated herein by reference. For example, the anti-VEGF antibody may be ranibizumab, bevacizumab, as well as antigen-binding variants thereof. Ranibizumab (molecular weight of 48 kD) is a humanized monoclonal Fab fragment directed against VEGF-A having the light and heavy chain variable domain sequences of Y0317 as described in SEQ ID Nos. 115 and 116 of U.S. Pat. No. 7,060,269. Ranibizumab inhibits endothelial cell proliferation and neovascularisation and may be used for the treatment of neovascular (wet) age-related macular degeneration (AMD), the treatment of visual impairment due to diabetic macular oedema (DME), the treatment of visual impairment due to macular oedema secondary to retinal vein occlusion (branch RVO or central RVO), or treatment of visual impairment due to choroidal neovascularisation (CNV) secondary to pathologic myopia. Bevacizumab (molecular weight of 149 kD) is likewise a full-length, humanized murine monoclonal antibody that recognizes all isoforms of VEGF and which is the parent antibody of ranibizumab. In another embodiment, the anti-VEGF antibody may also be a bispecific antibody that contains a first antigen binding site that binds to human vascular endothelial growth factor (e.g., VEGF-A) and a second antigen binding site that binds to human angiopoietin-2 (ANG-2). One example of such an anti-VEGF antibody is faricimab (molecular weight of 150 kD), which is described in WO 2019/154776 and WO 2014/009465 and is composed of an anti-Ang-2 antigen-binding fragment (Fab), an anti-VEGF-A Fab, and a modified fragment crystallizable region (Fc region).

Yet another suitable antibody is an anti-HER2 antibody. Such antibodies may be full length anti-HER2 antibodies; anti-HER2 antibody fragments having the same biological activity; including amino acid sequence variants and/or glycosylation variants of such antibodies or antibody fragments. Examples of humanized anti-HER2 antibodies include trastuzumab, pertuzumab, and margetuximab, as well as antigen-binding variants thereof. Yet other anti-HER2 antibodies with various properties have been described in Tagliabue et al., Int. J. Cancer, 47:933-937 (1991); McKenzie et al., Oncogene, 4:543-548 (1989); Cancer Res., 51:5361-5369 (1991); Bacus et al., Molecular Carcinogenesis, 3:350-362 (1990); Stancovski et al., PNAS (USA), 88:8691-8695 (1991); Bacus et al., Cancer Research, 52:2580-2589 (1992); Xu et al., Int. J. Cancer, 53:401-408 (1993); WO94/00136; Kasprzyk et al., Cancer Research, 52:2771-2776 (1992); Hancock et al., Cancer Res., 51:4575-4580 (1991); Shawver et al., Cancer Res., 54:1367-1373 (1994); Arteaga et al., Cancer Res., 54:3758-3765 (1994); Harwerth et al., J. Biol. Chem., 267:15160-15167 (1992); U.S. Pat. No. 5,783,186; and Klapper et al., Oncogene, 14:2099-2109 (1997).

Typically, the antibody is present in the formulation as a naked antibody. However, if desired, the antibody may also be conjugated with other agents, such as a cytotoxic agent. When used to treat cancer, for example, the immunoconjugate and/or antigen to which the antibody is bound may be internalized by the cell, resulting in increased therapeutic efficacy of the immunoconjugate in killing the cancer cell to which it binds. In one embodiment, the cytotoxic agent targets or interferes with nucleic acid in the cancer cell. Examples of such cytotoxic agents include maytansinoids, calioheamicins, ribonucleases and DNA endonucleases. Suitable immunoconjugates may include trastuzumab-maytansinoid immunoconjugates (T-DM1), such as T-MCC-DM1, the chemical name of which is N^{2′}-deacetyl-N^{2′}-(3-mercapto-1-oxopropyl)-maytansine-4-maleimidomethyl-cyclohexyl-1-carboxyl-trastuzumab.

In another particular embodiment, for example, the antibody may be an anti-CTLA-4 (cytotoxic T-lymphocyte associated protein-4) antibody, such as employed as immune checkpoint inhibitors for treating cancer. CLTA-4 is a protein receptor that downregulates the immune system. The term “CTLA-4” as used herein includes human CTLA-4 (hCTLA-4), variants, isoforms, and species homologs of hCTLA-4, and analogs having at least one common epitope with hCTLA-4. Specifically, CLTA-4 is an immunoglobulin cell surface receptor and an inhibitor of T cell activation and primarily expresses naive T cells after activation and FoxP3+ regulatory T cells (Tregs). T cell activation is dependent not only on T cell receptor (TCR) binding with an antigen presented via an Adenomatous polyposis coli (APC), but also in the presence of a costimulatory second signal, typically through binding of CD28 expressed on the T cell to CD80/86 found on the APC. Absences of this secondary signal may lead the T cell to recognize the presented peptide as a “self-antigen” or to develop tolerance to the antigen. CTLA-4 is a competitive homolog for CD28 that has a higher affinity to CD80 (B7-1), and to a lesser extent CD68 (B7-2) compared with CD28, leading to inhibition of T cell stimulation. TCR signaling immediately upregulates cell surface CTLA-4 expression, reaching peak expression at 2 to 3 days after activation, providing a negative feedback loop upon T cell activation. CTLA-4 within intracellular vesicles is also quickly transported to the immunologic synapse following T cell activation. At the immunologic synapse, CTLA-4 is stabilized by CD80/CD86 binding, allowing it to collect and inhibit CD28 binding. Accordingly, inclusion of an anti-CTLA-4 antibody can disrupt the inhibitory mechanism of CTLA-4. Antibodies that bind specifically to CLTA-4 have been described, for instance, in in U.S. Pat. Nos. 6,984,720 and 10,196,445.

In certain embodiments, the anti-CTLA-4 antibody can be Ipilimumab. Ipilimumab (also known as Yervoy®, designated MDX101) is a fully humanized IgG1 kappa immulgolubulin directed against CTLA-4. Ipilimumab has an approximate molecular weight of 148 kDa. Ipilimumab is produced in mammalian (Chinese hamster ovary) cell culture. Ipilimumab is a negative regulator of T-cell activity. Ipilimumab binds to CTLA-4 and blocks the interaction of CTLA-4 with its ligands, CD80 and CD86. Blockage of CTLA-4 by Ipilimumab has been shown to augment T-cell activation and proliferation, including the activation and proliferation of tumor infiltrating T-effector cells. Inhibition of CLTA-4 signaling can also reduce T-regulatory cell function, which may contribute to a general increase in T cell responsiveness, including the anti-tumor immune response.

Yet another suitable antibody is an anti-cKIT antibody. Such antibodies may be full length anti-cKIT antibodies; anti-cKIT antibody fragments having the same biological activity; including amino acid sequence variants and/or glycosylation variants of such antibodies or antibody fragments. cKIT is a single transmembrane, receptor tyrosine kinase inhibitor that binds the ligand stem cell factor (SCF). SCF induces homodimerization of cKIT, which activates its tyrosine kinase activity and signals through both the PI3-AKT and MAPK pathways as described in Kindblom et al., Am J. Path. 1998 152(5): 1259. Anti-cKIT antibodies with various properties are described in Gadd et al., Leuk. Res. 1985 (9): 1329, Broudy et al., Blood 1992 79(2):338, U.S. Pat. Nos. 8,552,157, and 9,540,443.

Another suitable antibody is an anti-4-1 BB antibody (anti-CD137). CD137 (4-1 BB) is a member of the tumor necrosis receptor (TNF-R) gene family, which includes proteins involved in regulation of cell proliferation, differentiation, and programmed cell death. Suitable anti-4-1 BB antibodies include urelumab (BMS-663513), which is a fully human IgG4 monoclonal antibody. Other suitable anti-4-1 BB antibodies are described in U.S. Pat. Nos. 7,288,638, 7,659,384, 8,137,667, 10,875,921, 11,242,395.

Typically, the antibody is present in the formulation as a naked antibody. However, if desired, the therapeutic agent can include an antibody drug conjugate (ADC). ADCs are a rapidly growing class of targeted therapeutics, represent a promising new approach toward improving both the selectivity and the cytotoxic activity of cancer drugs (e.g., cytotoxic agents). ADCs have three components: (1) a monoclonal antibody conjugated through a (2) linker to a (3) cytotoxin. The cytotoxins are attached to either lysine or cysteine sidechains on the antibody through linkers that react selectively with primary amines on lysine or with sulfhydryl groups on cysteine.

The maximum number of linkers/drugs that can be conjugated depends on the number of reactive amino or sulfhydryl groups that are present on the antibody. A typical antibody contains up to 90 lysines as potential conjugation sites; however, the typical number of cytotoxins per antibody for most ADCs is typically between 2 and 4 due to aggregation of ADCs with higher numbers of cytotoxins. As a result, conventional lysine linked ADCs are heterogeneous mixtures that contain from 0 to 10 cytotoxins per antibody conjugated to different amino groups on the antibody. Antibody cysteines can also be used for conjugation to cytotoxins through linkers that contain maleimides or other thiol specific functional groups. A typical antibody contains 4, or sometimes 5, interchain disulfide bonds (2 between the heavy chains and 2 between heavy and light chains) that covalently bond the heavy and light chains together and contribute to the stability of the antibodies in vivo. These interchain disulfides can be selectively reduced with dithiothreitol, tris(2-carboxyethyl)phosphine, or other mild reducing agents to afford 8 reactive sulfhydryl groups for conjugation. Cysteine linked ADCs are less heterogeneous than lysine linked ADCs because there are fewer potential conjugation sites; however, they also tend to be less stable due to partial loss of the interchain disulfide bonds during conjugation, since current cysteine linkers bond to only one sulfur atom. The typical number of cytotoxins per antibody for cysteine linked ADCs is also 2 to 4. For example, ADCETRIS is a heterogeneous mixture that contains 0 to 8 monomethylauristatin E residues per antibody conjugated through cysteines.

Key factors in the success of an ADC include that the monoclonal antibody is cancer antigen specific, non-immunogenic, low toxicity, and internalized by cancer cells; the cytotoxin is highly potent and is suitable for linker attachment; while the linker may be specific for cysteine (S) or lysine (N) binding, is stable in circulation, may be protease cleavable and/or pH sensitive, and is suitable for attachment to the cytotoxin. When used to treat cancer, for example, the ADC and/or antigen to which the antibody is bound may be internalized by the cell, resulting in increased therapeutic efficacy of the ADC in killing the cancer cell to which it binds.

The ADC can include at least one of the monoclonal antibodies described hereinabove with at least one cytotoxic agent (e.g., a chemotherapeutic agent as described hereinbelow). For instance, the ADC can comprise an anti-CLTA-4 antibody (e.g., ipilimumab) linked to one or more chemotherapeutic agents. In another example, the ADC can include an anti-PD1 antibody (e.g., nivolumab, pembrolizumab, or pidilizumab) linked to one or more chemotherapeutic agents. In another example, the ADC can include an anti-VEGF antibody (e.g., faricimab) linked to one or more chemotherapeutic agents. In another example, the ADC can include an anti-HER2 antibody (e.g., trastuzumab, pertuzumab, and margetuximab) linked to one or more chemotherapeutic agents. In yet another example, the ADC can include an anti-cKIT antibody linked to one or more chemotherapeutic agents. Suitable ADCs including anti-cKIT antibodies are described in PCT Publication No. WO 2014/150937.

The number of molecules of a drug (e.g., chemotherapeutic agent) conjugated per antibody is known as the drug-to-antibody ratio (DAR). The DAR can affect the potency and overall toxicity of the ADC. For instance, if the DAR is too low then the ACD may not be capable of providing therapeutically effective results, while if the DAR is too high, the patient may experience unwanted side effects. Given reaction and processing conditions, the DAR for ADS typically ranges anywhere from 0 to 15, such as from about 0 to 8. Accordingly, the ADCs as disclosed can have a DAR ranging from 0 to 15, such as 1 to 14, such as 2 to 13, such as 3 to 12, such as 4 to 11, such as 5 to 10, such as 6 to 9. In certain embodiments, the ADC has a DAR of 2 to 4.

The DAR for a manufacturing batch of ADC can be determined empirically using spectrophotometric measurements and ADC therapeutic compositions typically contain a mixture of ADC species that differ in drug load. Thus, the DAR for an ADC batch represents the average DAR of the ADC species within the batch. Varying DARs per batch contributes to potency variability. In order to reduce potency variability, the ADCs contained within a batch utilized in the present pharmaceutical formulation can include an average DAR of from about 2 to about 8, such as from about 2 to about 6, such as from about 2 to 4. In a certain example, the average DAR of the ADCs is about 3 to about 5, such as about 4.

Suitable FDA-approved ADCs for use in the pharmaceutical formulation of the present disclosure also include gemtuzumab ozogamicin (Mylotarg™), brentuximab vedotin (Adetris™), trastuzumab ematansine (Kadcyla™), inotuzumab ozogamicin (Besponsa™), moxetumomab pasudotox (Lumoxiti™), polatuzumab vedotin-piiq (Polivy™), enfortumab vedontin (Padcev™), trastuzumab deruxtecan (Enhertu™), sacituzumab govitecan (Trodelvy™), belantamab mafodotin-blmf (Blenrep™), locastuximab tesirine-lpyl (Zylonta™), tisotumab vedotin-tftv (Tivdak™), and combinations thereof. Other suitable ADCs include mirvetuximab soravtansin (IMGN853), transtuzumab duocarmazine (SYD985), depatuxizumab mafodotin (AGX-414), disitimab vedotin (RC48-ADC), and combinations thereof. Other suitable ADCs include Mirvetuximab soravtansine (IMGN853), Transtuzumab duocarmazine (SYD985), Depatuxizumab mafodotin (ABT-414), Disitimab vedotin (RC48-ADC), and combinations thereof. In other embodiments, the ADC can include an antibody that is linked to other types of molecules such as oligonucleotides, radionuclides, and protein toxins.

Antibodies of the present disclosure can also include radiolabeled antibodies. Such antibodies include a radioactive substance conjugated to the antibody configured to provide radioactivitiy directly to cancer cells. Suitable radiolabeled antibodies include ibritumomab tiuxetan (Zevalin™). Radiolabeled antibodies include any of the antibodies disclosed herein that are conjugated with a radioactive material, such as Yttrium-90. Other radiolabeled antibodies are described in International Patent Application No. WO 2009/0538203 and U.S. Pat. No. 7,402,385.

Antibodies of the present disclosure can also include multispecific antibodies, such as bispecific antibodies (BsAbs). Multispecific antibodies refers to any antibody that can bind simultaneously to two or more different antigens, while BsAbs refers to an antibody that can bind simultaneously to two different antigens. One example BsAb is a bispecific T cell engager (BiTE) with one arm targeting CD3 on T cells and the other recognizing target proteins on tumor cells, thereby activating the T cells to kill the tumor cells. In addition to their interaction with T cells, BsABs have also been designed to engage other effector ells, such as natural killer (NK) cells and macrophages for cancer therapy. Suitable BsAbs include blinatumomab (BLINCYTO™) which targets both CD19 and CD3 and catumaxomab (Removab™) which targets human EpCAM and human CD3 receptors. Other suitable BsAbs include AMG 330 (anti-CD3/CD33), AMG 427 (anti-CD3/FLT3), AMG 673 (anti-CD3/CD33), AMG 701 (anti-CD3/BCMA), AMG 160 (anti-CD3/prostate specific membrane antigen (PSMA)), AMG 596 (anti-CD3/epidermal growth factor receptor (EGFR) and AMG 757 (anti-CD3/DLL-3), all developed by Amgen.

Other suitable BsABs or multispecific antibodies can include those currently FDA-approved and/or under clinical trial testing including Blinatumomab/Blincyto/MT103/MEDI-538/AMG103 (clinical trials identifiers NCT01466179 NCT02013167), AFM11 (clinical trials identifier NCT02848911), AMG562 (clinical trials identifier NCT03571828), REGN1979 (clinical trials identifier NCT03888105), Glofitamab/R07082859 (clinical trials identifier NCT03075696), Plamotamab/XmAb13676 (clinical trials identifier NCT02924402), Mosunetuzumab/RG7828/RO703081 (clinical trials identifier NCT04313608), GEN3013 (clinical trials identifier NCT03625037), AMG673 (clinical trials identifier NCT03224819), AMV-564 (clinical trials identifier NCT03144245), ISB 1342 (clinical trials identifier NCT03309111), JNJ-63709178 (clinical trials identifier NCT02715011), SAR440234 (clinical trials identifier NCT03594955), Vibecotamab/Xmab14045 (clinical trials identifier NCT02730312), AMG420/BI 836909 (clinical trials identifier NCT03836053), CC-93269/EM801 (clinical trials identifier NCT03486067), Teclistamab/JNJ-64007957 (clinical trials identifier NCT04557098), PF-06863135 (clinical trials identifier NCT04649359), REGN5458 (clinical trials identifier NCT03761108), Catumaxomab/removab (clinical trials identifier NCT03070392), RG6194/BTRC4017A (clinical trials identifier NCT03448042), M802 (clinical trials identifier NCT04501770), GBR1302 (clinical trials identifier NCT03983395), Cibisatamab/RG7802/RO6958688 (clinical trials identifier NCT03866239), AMG211 (clinical trials identifier NCT02291614), AMG160 (clinical trials identifier NCT03792841), MOR209/ES414 (clinical trials identifier NCT02262910), Pasotuxizumab/BAY2010112 (clinical trials identifier NCT01723475), REGN5678 (clinical trials identifier NCT03972657), FS120 (clinical trials identifier NCT04648202), PRS-343 (clinical trials identifier NCT03330561), AFM13 (clinical trials identifiers NCT03192202, NCT04101331), AFM24 (clinical trials identifier NCT04259450), GTB-3550, OXS-35504 (clinical trials identifier NCT03214666), MED15752 (clinical trials identifier NCT04522323), AK104 (clinical trials identifier NCT04172454), XmAb20717 (clinical trials identifier NCT03517488), MGD019 (clinical trials identifier NCT03761017), MGD013 (clinical trials identifier NCT03219268), R07121661, RG7769 (clinical trials identifier NCT03708328), KN046 (clinical trials identifiers NCT03872791, NCT04474119, NCT04469725, NCT03733951), FS118 (clinical trials identifier NCT03440437), LY3415244 (clinical trials identifier NCT03752177), IBI318/LY3434172 (clinical trials identifier NCT03875157), IBI315 IBI318/LY3434172 (clinical trials identifier NCT04162327), AK112 (clinical trials identifier NCT04047290), IB1319 (clinical trials identifier NCT04708210), FS222 (clinical trials identifier NCT04740424), MCLA-145 (clinical trials identifier NCT03922204), ATOR 1015 (clinical trials identifier NCT03782467), XmAb23104 (clinical trials identifier NCT03752398), TG-1801/NI-1701 (clinical trials identifier NCT03804996), IMM0306 (clinical trials identifier CTR20192612), IB1322 (clinical trials identifiers NCT04338659, NCT04328831), HX009 (clinical trials identifier NCT04097769), JNJ-61186372/Amivantamab (clinical trials identifier NCT02609776), MCLA-158 (clinical trials identifier NCT03526835), MCLA-128/Zenocutuzumab (clinical trials identifier NCT03321981), KN026 (clinical trials identifier NCT04521179), MBS301 (clinical trials identifier NCT03842085), ZW25 (clinical trials identifier NCT02892123), ZW49 (clinical trials identifier NCT03821233), MM-141 (clinical trials identifier NCT02399137), BI 836880 (clinical trials identifiers NCT03972150, NCT03697304), R05520985/Vanucizumab (clinical trials identifier NCT02141295), ABT-165/Dilpacimab (clinical trials identifier NCT01946074), OMP-305B83/Navicixizumab (clinical trials identifier NCT03030287), RG7386/RO6874813 (clinical trials identifier NCT02558140), OXS-1550/DT2219ARL (clinical trials identifier NCT02370160), and combinations thereof.

As noted above, the antibodies can include antibody fragments (Fabs). Such FDA-approved Fabs include Ranibizumab (Lucentis™), Abciximab (ReoPro™), Certolizumab pegol (Cimzia™), Idarucizumab (Praxbind™), Digoxin Immune Fab (DigiFab™), crotalidae-polyvalent immune fab (CroFab™) arotalidae-polyvalent immune Fab (Anavip™), centruroides immune F(ab′)2 (Anascorp™), Brolucizumab (Beovu™), Caplacizumab (Cablivi™), or combinations thereof. Additionally Fabs that can be utilized in the present device also include Copper Cu 64-DOTA-B-Fab (clinical trials identifier NCT02708511), CSR02-Fab-TF (clinical trials identifier NCT04601428), Ranibizumab (clinical trials identifier NCT00540930), Naptumomab estafenatox (clinical trials identifier NCT00420888), IMCgp100 (clinical trials identifier NCT01209676), L19-IL2 (clinical trials identifier NCT02086721), rM28 (clinical trials identifier NCT00204594), D2C7-IT (clinical trials identifier NCT02303678), NM21-1480 (clinical trials identifier NCT04442126), Vicinium (clinical trials identifier NCT02449239), [124 I] PSCA-Minibody (clinical trials identifier NCT02092948), 6B111-OCIK (clinical trials identifier NCT03542669), T84.66 (clinical trials identifier NCT00647153), BCMA VHH CAR-T Cell (clinical trials identifier NCT03664661), CD19/20 bispecific VHH-derived CAR-T Cells (clinical trials identifier NCT03881761), ALX-0651 (clinical trials identifier NCT01374503), αPD1-MSLN-CAR T cells (clinical trials identifiers NCT04489862, NCT04503980), [131I]-SGMIB anti-HER2 VHH1 (clinical trials identifier NCT02683083), 68-Ga NOTA-anti-MMR-VHH2 (clinical trials identifier NCT04168528), 68-GaNOTA-anti-HER2 VHH1 VHH (clinical trials identifiers NCT03924466, NCT03331601), 99mTc-MIRC208 (clinical trials identifier NCT04591652), TAS226 (clinical trials identifier NCT01529307), and combinations thereof.

In embodiments, the antibodies can include antibody cytokine fusion proteins fusion proteins, referred to as immunocytokines. Cytokines constitute a broad and loosely defined class of relatively small proteins that regulate the immune response. The systemic administration of proinflammatory cytokines is often associated with severe off target toxicity, particularly flu-like symptoms, which may limit the dose and prevent the escalation of dosages needed for developing therapeutically effective regimens. Similar to ADCs, utilization of cytokines with antibodies or antibody fragments as vehicles has been used for the targeted delivery of immunomodulatory cytokines (such as interleukin (IL)-2, IL-12, and tumor necrosis factor (TNF) including TNFa and TNFb) to leverage the local tumor microenvironment (TME) and activate anticancer immune responses. Suitable immunocytokines that may be used in accordance with the present disclosure include, but are not limited to, L19IL2 (clinical trials identifier NCT01058538), L19TNFa (clinical trials identifier NCT01253837), F16IL2 (clinical trials identifier NCT01134250), hu14.18-IL2 (clinical trials identifier NCT00003750), huKS-IL2 (EMD 273066) (clinical trials identifier NCT00132522), DI-Leu16-IL2 (anti-CD20-IL2) (clinical trials identifier NCT01374288), NHS-IL12 (clinical trials identifier NCT04303117), NHS-IL2-LT (EMD 521873) (clinical trials identifier NCT00879866), Anti-CEA-IL2v (cergutuzumab amunaleukin) (clinical trials identifier NCT02350673), and combinations thereof.

Antibodies of the present disclosure also include antibody-small interfering RNA (siRNA) conjugates (ARCs). Since antibodies show high specificity toward overexpressed antigens in certain cell types or tissues, antibodies can be utilized to target delivery of siRNA molecules. siRNA molecules can be linked to the antibodies with covalent linkages with lysine or cysteine residues. Accordingly, antibodies utilized herein can include the disclosed antibodies linked with one or more siRNA molecules for treating cancer. Such ARCs can be used to treat a variety of cancers. Utilization of ARCs to treat cancer has had a few drawbacks, namely ARCs may not readily enter the cell due to the negative charge of the appended siRNA, which makes it difficult to overcome the thermodynamic barriers presented by the cell membrane. As such, ARCs can be incorporated into the device of the present disclosure and released within the tumor cells themselves, eliminating thermodynamic barriers at the cell membrane.

Regardless of the type of antibody employed, it is generally stable at high enough temperatures so that it can be incorporated into the polymer matrix at or near the melting temperature of the polymer matrix without significantly degrading (e.g., melting) during manufacturing or use of the device. For example, the antibody may remain stable at temperatures of from about 20° C. to about 100° C., in some embodiments from about 25° C. to about 80° C., in some embodiments from about 30° C. to about 70° C., in some embodiments from about 35° C. to about 65° C., and in some embodiments, from about 40° C. to about 60° C. The antibody may be inherently stable at such temperatures, or it may also be encapsulated or otherwise protected by a carrier component that is stable at such temperatures, such as a carrier component containing peptides, proteins, carbohydrates (e.g., sugars), polymers, lipids, etc.

In certain embodiments, the pharmaceutical formulation may only contain antibodies (e.g., one antibody) as a therapeutic agent. Of course, in other embodiments, the formulation may contain an antibody in combination with one or more additional types of therapeutic agents. Such additional therapeutic agents may be a macromolecular compound having a relatively large molecular weight, such as about 1 kilodaltons (kDa) or more, in some embodiments from about 2 kDa to about 1000 kDa, in some embodiments from about 20 kDa to about 950 kDa, in some embodiments from about 50 kDa to about 750 kDa, and in some embodiments, from about 100 kDa to about 500 kDa, or any range therebetween. The macromolecular compound may, for instance, include a protein, peptide, enzyme, antibody, interferon, interleukin, blood factor, vaccine, nucleotide, lipid, or an analogue, derivative, or combination thereof. Alternatively, small molecule therapeutic agents may also be employed, such as those having a molecular weight of less than about 1,000 Da, in some embodiments about 900 Da or less, in some embodiments from about 10 to about 800 Da, and in some embodiments, from about 20 to about 700 Da, or any range therebetween.

Particular examples of suitable additional therapeutic agents may include, for instance, proteins, peptides, enzymes, interferons, interleukins, blood factors, vaccines, nucleotides, lipids, small molecule drug compounds, etc., as well as analogues, derivatizes, and combinations thereof. For instance, the therapeutic agent may include non-steroidal anti-inflammatories (e.g., salicylate, indomethacin, ibuprofen, diclofenac, flurbiprofen, piroxicam); antiallergenics (e.g., sodium chromoglycate, antazoline, methapyriline, chlorpheniramine, cetrizine, pyrilamine, prophenpyridamine); anti-proliferative agents (e.g., 1,3-cis retinoic acid); decongestants (e.g., phenylephrine, naphazoline, tetrahydrazoline); miotics and anti-cholinesterase (e.g., pilocarpine, salicylate, carbachol, acetylcholine chloride, physostigmine, eserine, diisopropyl fluorophosphate, phospholine iodine, demecarium bromide); antineoplastics (e.g., carmustine, cisplatin, fluorouracil); immunological drugs (e.g., vaccines and immune stimulants); hormonal agents (e.g., estrogens, estradiol, progestational, progesterone, insulin, calcitonin, parathyroid hormone, peptide and vasopressin hypothalamus releasing factor); immunosuppressive agents, growth hormone antagonists, growth factors (e.g., epidermal growth factor, fibroblast growth factor, platelet derived growth factor, transforming growth factor beta, somatotropin, fibronectin); inhibitors of angiogenesis (e.g., angiostatin, anecortave acetate, thrombospondin, etc.); interferons (e.g., interferon alpha-2b, peg interferon alpha-2a, interferon alpha-2b+ribavirin, pegylated interferon-2a, interferon beta-1a, interferon beta); interleukins (e.g., interleukin-2); vaccines (e.g., whole viral particles, recombinant proteins, subunit proteins, gp41, gp120, gp140, DNA vaccines, plasmids, bacterial vaccines, polysaccharides, extracellular capsular polysaccharides); dopamine agonists; radiotherapeutic agents; peptides; proteins; enzymes; extracellular matrix components; ACE inhibitors; free radical scavengers; chelators; antioxidants; anti-polymerases; photodynamic therapy agents; gene therapy agents; corticosteroids (e.g., dexamethasone), tyrosine kinase inhibitors (e.g., axitinib, bosutinib, cabozantinib, crizotinib, dasatinib, erlotinib, gefitinib, imatinib, lapatinib, nilotinib, pazopanib, ponatinib, ruxolitinib, sorafenib, sunitinib, vatalanib, vemurafenib, etc.), and so forth, as well as combinations of any of the foregoing.

B. Excipients

In certain embodiments, the pharmaceutical formulation may contain only therapeutic agents (e.g., antibodies). Because therapeutic agents are generally water-soluble, the relative amount of such agents may be selectively controlled to help achieve the desired release rate. Namely, when only therapeutic agents are present, the weight ratio of the therapeutic agents to the polymer matrix is typically controlled within a range of from about 0.7 to about 1, in some embodiments from about 0.75 to about 1, in some embodiments from about 0.75 to about 0.9, and in some embodiments, from about 0.8 to about 0.85, and the therapeutic agent(s) may likewise constitute from about 40 wt. % to about 50 wt. %, in some embodiments from about 41 wt. % to about 48 wt. %, and in some embodiments, from about 42 wt. % to about 47 wt. % of the device.

Of course, as indicated above, the pharmaceutical formulation may also optionally contain one or more excipients, such as buffering agents, saccharides (e.g., sugars, sugar alcohols, etc.), surfactants, antimicrobial agents, preservatives, cell permeability enhancers, etc., to enhance properties and processability. Because these excipients are generally water-soluble, the relative amount of such excipients may also be selectively controlled to help achieve the desired release rate. For example, when excipients are present, the weight ratio of the therapeutic agents to the polymer matrix may be from about 0.3 to about 1, in some embodiments from about 0.75 to about 1, in some embodiments from about 0.35 to about 0.5, and in some embodiments, from about 0.38 to about 0.45. Likewise, the optional excipient(s) may constitute from about 10 wt. % to about 80 wt. %, and in some embodiments, from about 20 wt. % to about 70 wt. %, and in some embodiments, from about 40 wt. % to about 60 wt. % of the pharmaceutical formulation, and therapeutic agent(s) (e.g., antibodies) may likewise constitute 20 wt. % to about 90 wt. %, and in some embodiments, from about 30 wt. % to about 80 wt. %, and in some embodiments, from about 40 wt. % to about 60 wt. % of the pharmaceutical formulation.

The pH of the pharmaceutical formulation may, for example, desirably be within a range of from about 5.5 to about 7.5, and in some embodiments, from about 5.7 to about 6.3. To help achieve the desired level, one or more buffering agents may be employed. Exemplary buffering agents may include, for instance, a histidine buffer, such as L-histidine, L-histidine hydrochloride, L-histidine hydrochloride monohydrate, histidine succinate, histidine acetate, histidine citrate, histidine chloride or histidine sulfate, etc., as well as combinations thereof. Other suitable buffering agents may include, for instance, a salt of a metal (e.g., sodium) and an acid, such as a carboxylic acid (e.g., acetic acid, succinic acid, citric acid, etc.), phosphoric acid, etc. Such salts may include, for instance, sodium succinate, sodium acetate, sodium acetate/acetic acid, sodium phosphate, etc. When employed, the optional buffering agent(s) may constitute from about 0.01 wt. % to about 20 wt. %, and in some embodiments, from about 0.1 wt. % to about 10 wt. %, and in some embodiments, from about 0.5 wt. % to about 5 wt. % of the pharmaceutical formulation.

If desired, one or more saccharides may also be employed in the formulation to help act as a degradation stabilizer for the antibodies. Particularly suitable saccharides include, for instance, monosaccharides (e.g., dextrose, fructose, galactose, ribose, deoxyribose, etc.); disaccharides (e.g., sucrose, lactose, maltose, trehalose, etc.); sugar alcohols (e.g., xylitol, sorbitol, mannitol, maltitol, erythritol, galactitol, inositol, lactitol, etc.); and so forth, as well as combinations thereof. Trehalose (e.g., α,α-trehalose or α,α-trehalose dihydrate) is particularly suitable for use in the formulation. When employed, saccharides may constitute from about 10 wt. % to about 70 wt. %, and in some embodiments, from about 20 wt. % to about 65 wt. %, and in some embodiments, from about 30 wt. % to about 60 wt. % of the pharmaceutical formulation.

Surfactants may also be employed as an optional stabilizer for the formulation. Nonionic surfactants, which typically have a hydrophobic base (e.g., long chain alkyl group or an alkylated aryl group) and a hydrophilic chain (e.g., chain containing ethoxy and/or propoxy moieties), are particularly suitable. Some suitable nonionic surfactants that may be used include, but are not limited to, ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C8-C18) acids, condensation products of ethylene oxide with long chain amines or amides, condensation products of ethylene oxide with alcohols, fatty acid esters, monoglyceride or diglycerides of long chain alcohols, and mixtures thereof. Particularly suitable nonionic surfactants may include ethylene oxide condensates of fatty alcohols, polyoxyethylene ethers of fatty acids, polyoxyethylene sorbitan fatty acid esters, and sorbitan fatty acid esters, etc. The fatty components used to form such emulsifiers may be saturated or unsaturated, substituted or unsubstituted, and may contain from 6 to 22 carbon atoms, in some embodiments from 8 to 18 carbon atoms, and in some embodiments, from 10 to 14 carbon atoms. Sorbitan fatty acid esters (e.g., monoesters, diester, triesters, etc.) that have been modified with polyoxyethylene are one particularly useful group of nonionic surfactants. These materials are typically prepared through the addition of ethylene oxide to a 1,4-sorbitan ester. The addition of polyoxyethylene converts the lipophilic sorbitan ester surfactant to a hydrophilic surfactant that is generally soluble or dispersible in water. Examples of such materials are commercially available under the designation TWEEN® (e.g., TWEEN® 20, or polyethylene (20) sorbitan monoolaurate). When employed, surfactants may constitute from about 0.001 wt. % to about 5 wt. %, and in some embodiments, from about 0.01 wt. % to about 2 wt. %, and in some embodiments, from about 0.05 wt. % to about 1 wt. % of the solids content of the pharmaceutical formulation.

If desired, the pharmaceutical formulation may be antibody formulations that are commercially available for the treatment of certain conditions. For example, one suitable pharmaceutical formulation may be HERCEPTIN™, which contains an anti-HER2 monoclonal antibody (trastuzumab) in combination with α,α-trehalose dihydrate, L-histidine, L-histidine hydrochloride, and polyethylene (20) sorbitan monolaurate. Another suitable formulation may be AVASTIN™, which contains an anti-VEGF monoclonal antibody (bevacizumab) in combination with trehalose dihydrate, sodium phosphate, and polyethylene (20) sorbitan monolaurate. Similarly, yet another suitable formulation may be XOLAIR™, which contains an anti-IgE monoclonal antibody (omalizumab) in combination with sucrose, histidine, histidine hydrochloride monohydrate, and polyethylene (20) sorbitan monolaurate.

Regardless of any optional excipients employed, it is generally desired that the pharmaceutical formulation is in the form of a dehydrated particulate material prior to being incorporated into the polymer matrix. In certain embodiments, for example, a liquid formulation may be dehydrated under reduced pressure using standard freeze-drying equipment or an equivalent apparatus. The antibody and and/or other excipients may also be frozen in liquid nitrogen before dehydration and then placed under reduced pressure. Spray drying may also be employed to form such a particulate material. In addition, various techniques (e.g., electrostatic approaches) can be employed to atomize droplets and allow for lower temperatures to be used.

### III. Implantable Device

The implantable device may be formed through a variety of known techniques, such as by hot-melt extrusion, injection molding, solvent casting, dip coating, spray coating, microextrusion, coacervation, etc. In one embodiment, a hot-melt extrusion technique may be employed. Hot-melt extrusion is generally a solvent-free process in which the components of the device (e.g., hydrophobic polymer(s), antibodies, optional excipients, etc.) may be melt blended and optionally shaped in a continuous manufacturing process to enable consistent output quality at high throughput rates. This technique is particularly well suited to certain types of hydrophobic polymers (e.g., ethylene vinyl acetate copolymers) that can exhibit a high degree of long-chain branching with a broad molecular weight distribution. This combination of traits can lead to shear thinning of the copolymer during the extrusion process, which help facilitates hot-melt extrusion. Furthermore, the polar comonomer units can serve as an “internal” plasticizer by inhibiting crystallization of the olefin chain segments. This may lead to a lower melting point of the polymer, which further enhances its ability to be processed with the antibody.

During a hot-melt extrusion process, melt blending generally occurs at a temperature that is similar to or even less than the melting temperature of the antibodies. Melt blending may also occur at a temperature that is similar to or slightly above the melting temperature of the hydrophobic polymer(s). The ratio of the melt blending temperature to the degradation temperature of the antibody may, for instance, be about 2 or less, in some embodiments about 1.8 or less, in some embodiments from about 0.1 to about 1.6, in some embodiments from about 0.2 to about 1.5, and in some embodiments, from about 0.4 to about 1.2. The melt blending temperature may, for example, be from about 30° C. to about 100° C., in some embodiments, from about 40° C. to about 80° C., and in some embodiments, from about 50° C. to about 70° C. Any of a variety of melt blending techniques may generally be employed. For example, the components may be supplied separately or in combination to an extruder that includes at least one screw rotatably mounted and received within a barrel (e.g., cylindrical barrel). The extruder may be a single screw or twin screw extruder. For example, one embodiment of a single screw extruder may contain a housing or barrel and a screw rotatably driven on one end by a suitable drive (typically including a motor and gearbox). If desired, a twin-screw extruder may be employed that contains two separate screws. The configuration of the screw is not particularly critical and it may contain any number and/or orientation of threads and channels as is known in the art. For example, the screw typically contains a thread that forms a generally helical channel radially extending around the center of the screw. A feed section and melt section may be defined along the length of the screw. The feed section is the input portion of the barrel where the hydrophobic polymer(s) and/or antibody are added. The melt section is the phase change section in which the copolymer is changed from a solid to a liquid-like state. While there is no precisely defined delineation of these sections when the extruder is manufactured, it is well within the ordinary skill of those in this art to reliably identify the feed section and the melt section in which phase change from solid to liquid is occurring. Although not necessarily required, the extruder may also have a mixing section that is located adjacent to the output end of the barrel and downstream from the melting section. If desired, one or more distributive and/or dispersive mixing elements may be employed within the mixing and/or melting sections of the extruder. Suitable distributive mixers for single screw extruders may include, for instance, Saxon, Dulmage, Cavity Transfer mixers, etc. Likewise, suitable dispersive mixers may include Blister ring, Leroy/Maddock, CRD mixers, etc. As is well known in the art, the mixing may be further improved by using pins in the barrel that create a folding and reorientation of the polymer melt, such as those used in Buss Kneader extruders, Cavity Transfer mixers, and Vortex Intermeshing Pin mixers.

If desired, the ratio of the length (“L”) to diameter (“D”) of the screw may be selected to achieve an optimum balance between throughput and blending of the components. The L/D value may, for instance, range from about 10 to about 50, in some embodiments from about 15 to about 45, and in some embodiments from about 20 to about 40. The length of the screw may, for instance, range from about 0.1 to about 5 meters, in some embodiments from about 0.4 to about 4 meters, and in some embodiments, from about 0.5 to about 2 meters. The diameter of the screw may likewise be from about 5 to about 150 millimeters, in some embodiments from about 10 to about 120 millimeters, and in some embodiments, from about 20 to about 80 millimeters. In addition to the length and diameter, other aspects of the extruder may also be selected to help achieve the desired degree of blending. For example, the speed of the screw may be selected to achieve the desired residence time, shear rate, melt processing temperature, etc. For example, the screw speed may range from about 10 to about 800 revolutions per minute (“rpm”), in some embodiments from about 20 to about 500 rpm, and in some embodiments, from about 30 to about 400 rpm. The apparent shear rate during melt blending may also range from about 100 seconds⁻¹ to about 10,000 seconds⁻¹, in some embodiments from about 500 seconds⁻¹ to about 5000 seconds⁻¹, and in some embodiments, from about 800 seconds⁻¹ to about 1200 seconds⁻¹. The apparent shear rate is equal to 4Q/πR³, where Q is the volumetric flow rate (“m³/s”) of the polymer melt and R is the radius (“m”) of the capillary (e.g., extruder die) through which the melted polymer flows.

Once melt blended together, the resulting polymer composition may be extruded through an orifice (e.g., die) and formed into pellets, sheets, fibers, filaments, etc., which may be thereafter shaped into the implantable device using a variety of known shaping techniques, such as injection molding, compression molding, nanomolding, overmolding, blow molding, three-dimensional printing, etc. Injection molding may, for example, occur in two main phases—i.e., an injection phase and holding phase. During the injection phase, a mold cavity is filled with the molten polymer composition. The holding phase is initiated after completion of the injection phase in which the holding pressure is controlled to pack additional material into the cavity and compensate for volumetric shrinkage that occurs during cooling. After the shot has built, it can then be cooled. Once cooling is complete, the molding cycle is completed when the mold opens and the part is ejected, such as with the assistance of ejector pins within the mold. Any suitable injection molding equipment may generally be employed in the present invention. In one embodiment, an injection molding apparatus may be employed that includes a first mold base and a second mold base, which together define a mold cavity having the shape of the implantable device. The molding apparatus includes a resin flow path that extends from an outer exterior surface of the first mold half through a sprue to a mold cavity. The polymer composition may be supplied to the resin flow path using a variety of techniques. For example, the composition may be supplied (e.g., in the form of pellets) to a feed hopper attached to an extruder barrel that contains a rotating screw (not shown). As the screw rotates, the pellets are moved forward and undergo pressure and friction, which generates heat to melt the pellets. A cooling mechanism may also be provided to solidify the resin into the desired shape for the implantable device (e.g., disc, rod, etc.) within the mold cavity. For instance, the mold bases may include one or more cooling lines through which a cooling medium flows to impart the desired mold temperature to the surface of the mold bases for solidifying the molten material. The mold temperature (e.g., temperature of a surface of the mold) may range from about 50° C. to about 120° C., in some embodiments from about 60° C. to about 110° C., and in some embodiments, from about 70° C. to about 90° C.

As indicated above, another suitable technique for forming an implantable device of a desired shape and size is three-dimensional printing. During this process, the polymer composition may be incorporated into a printer cartridge that is readily adapted for use with a printer system. The printer cartridge may, for example, contains a spool or other similar device that carries the polymer composition. When supplied in the form of filaments, for example, the spool may have a generally cylindrical rim about which the filaments are wound. The spool may likewise define a bore or spindle that allows it to be readily mounted to the printer during use. Any of a variety of three-dimensional printer systems can be employed in the present invention. Particularly suitable printer systems are extrusion-based systems, which are often referred to as “fused deposition modeling” systems. For example, the polymer composition may be supplied to a build chamber of a print head that contains a platen and gantry. The platen may move along a vertical z-axis based on signals provided from a computer-operated controller. The gantry is a guide rail system that may be configured to move the print head in a horizontal x-y plane within the build chamber based on signals provided from controller. The print head is supported by the gantry and is configured for printing the build structure on the platen in a layer-by-layer manner, based on signals provided from the controller. For example, the print head may be a dual-tip extrusion head.

Compression molding (e.g., vacuum compression molding) may also be employed. In such a method, a layer of the device may be formed by heating and compressing the polymer compression into the desired shape while under vacuum. More particularly, the process may include forming the polymer composition into a precursor that fits within a chamber of a compression mold, heating the precursor, and compression molding the precursor into the desired layer while the precursor is heated. The polymer composition may be formed into a precursor through various techniques, such as by dry power mixing, extrusion, etc. The temperature during compression may range from about 50° C. to about 120° C., in some embodiments from about 60° C. to about 110° C., and in some embodiments, from about 70° C. to about 90° C. A vacuum source may also apply a negative pressure to the precursor during molding to help ensure that it retains a precise shape. Examples of such compression molding techniques are described, for instance, in U.S. Pat. No. 10,625,444 to Treffer, et al., which is incorporated herein in its entirety by reference thereto.

The resulting implantable medical device may have a variety of different geometric shapes, such as cylindrical (rod), disc, ring, doughnut, helical, elliptical, triangular, ovular, etc. In one embodiment, for example, the implantable medical device may have a generally circular cross-sectional so that the overall structure is in the form of a cylinder (rod) or disc. The implantable medical device also has a relatively small size, such as a thickness (e.g., diameter) of from about 0.1 to about 10 millimeters, in some embodiments from about 0.1 to about 5 millimeters, in some embodiments from about 0.3 to about 2 millimeters, and in some embodiments, from about 0.4 to about 0.8 millimeters. The length of the implantable medical device may vary, but is typically from about 1 to about 250 millimeters, in some embodiments from about 2 to about 200 millimeters, in some embodiments from about 10 to about 150 millimeters, and in some embodiments, from about 20 to about 100 millimeters.

### IV. Use of Device

The implantable device can be effective for sustained release of an antibody over a prolonged period of time such as noted above. Of course, the actual dosage level of the antibody delivered will vary depending on the particular antibody employed and the time period for which it is intended to be released. The dosage level is generally high enough to provide a therapeutically effective amount of the antibody to render a desired therapeutic outcome, i.e., a level or amount effective to reduce or alleviate symptoms of the condition for which it is administered. More particularly, a used herein, the phrase “therapeutically effective amount” means a dose of the antibody that results in a detectable improvement in one or more symptoms of a disorder, or a dose of antibody that inhibits, prevents, lessens, or delays the progression of a disorder. The exact amount necessary will vary, depending on the subject being treated, the age and general condition of the subject to which the antibody is to be delivered, the capacity of the subject's immune system, the degree of effect desired, the severity of the condition being treated, the particular antibody selected and mode of administration of the composition, among other factors. In one embodiment, for example, a therapeutically effective amount can be from about 0.05 mg to about 5 mg, in some embodiments from about 0.1 mg to about 4 mg, and in some embodiments, from about 0.5 to about 3 mg. The amount of the antibody contained within the individual doses may be expressed in terms of milligrams of drug per kilogram of patient body weight (i.e., mg/kg). For example, the antibody may be administered to a patient at a dose of about 0.0001 to about 10 mg/kg of patient body weight.

The device may be implanted subcutaneously, orally, mucosally, etc., using standard techniques. The delivery route may be intrapulmonary, gastroenteral, subcutaneous, intramuscular, or for introduction into the central nervous system, intraperitoneum or for intraorgan delivery. The implantable device may be suitable for delivering an antibody to treat a wide variety of conditions, such as cancer, allergies, inflammation, immunologically-mediated diseases, metabolic diseases, eye disorders (e.g., angiogenic eye disorders), etc. The device may be placed in a tissue site of a patient in, on, adjacent to, or near a tumor, such as a tumor of the pancreas, billary system, gallbladder, liver, small bowel, colon, brain, lung, eye, etc.

In one embodiment, the implantable device may release an antibody that can treat cancer. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer. In one specific embodiment, the implantable device may deliver an anti-HER2 antibody to treat a cancer that “overexpresses” a HER receptor. Such cancers include those that have significantly higher levels of a HER receptor, such as HER2, at the cell surface thereof, compared to a noncancerous cell of the same tissue type. Such overexpression may be caused by gene amplification or by increased transcription or translation. If desired, treatment may include a combination of the antibody formulation and a chemotherapeutic agent. The combined administration includes co-administration or concurrent administration, using separate formulations or a single pharmaceutical formulation, and consecutive administration in either order, wherein preferably there is a time period while both (or all) active agents simultaneously exert their biological activities. Thus, the chemotherapeutic agent may be administered prior to, or following, administration of the antibody formulation in accordance with the present invention. In this embodiment, the timing between at least one administration of the chemotherapeutic agent and at least one administration of the antibody formulation in accordance with the present invention is preferably approximately 1 month or less, and most preferably approximately 2 weeks or less. Alternatively, the chemotherapeutic agent and the antibody formulation may be administered concurrently to the patient, in a single formulation or separate formulations.

The implantable device may also be suitable for treatment of an angiogenic eye disorder, which includes any disease of the eye which is caused by or associated with the growth or proliferation of blood vessels or by blood vessel leakage. Non-limiting examples of angiogenic eye disorders that are treatable using the methods of the present invention include age-related macular degeneration (e.g., wet AMD, exudative AMD, etc.), retinal vein occlusion (RVO), central retinal vein occlusion (CRVO; e.g., macular edema following CRVO), branch retinal vein occlusion (BRVO), diabetic macular edema (DME), choroidal neovascularization (CNV; e.g., myopic CNV), iris neovascularization, neovascular glaucoma, post-surgical fibrosis in glaucoma, proliferative vitreoretinopathy (PVR), optic disc neovascularization, corneal neovascularization, retinal neovascularization, vitreal neovascularization, pannus, pterygium, vascular retinopathy, and diabetic retinopathies.

The manner in which the device of the present invention is implanted within the eye of a patient may vary as known to those skilled in the art. For example, the device may be inserted into the anterior segment (anterior chamber between the posterior surface of the cornea and the iris and/or the posterior chamber between the iris and front face of the vitreous humor), the posterior segment (e.g., anterior hyaloid membrane, vitreous humor, retina, choroid, etc.), the suprachoroidal space, or a combination thereof. When treating inflammatory eye conditions, such as uveitis, infection corneal ulcers, endophthalmitis, keratitis, conjunctivitis, and thyroid eye disease, it is generally desired that the therapeutic agent is delivered to the posterior segment. To avoid having to pass through the blood-aqueous barrier, the implantable device may be directly inserted into the posterior segment, such as into the vitreous humor (“intravitreally”). Intravitreal injection techniques are well known in the art any may include, for instance, the use of a hollow needle through which the implant is passed. The needle may have a small diameter size (e.g., 18 to 30 gauge needle) so that the incision is self sealing and the implantation occurs in a closed chamber. A self-sealing incision may also be formed using a conventional “tunneling” procedure in which a spatula-shaped scalpel is used to create a generally inverted V-shaped incision through the cornea. In a preferred mode, the instrument used to form the incision through the cornea remains in place (that is, extends through the corneal incision) during the procedure and is not removed until after implantation. Such incision-forming instrument either may be used to place the ocular implant or may cooperate with a delivery instrument to allow implantation through the same incision without withdrawing the incision-forming instrument. Of course, in other modes, various surgical instruments may be passed through one or more corneal incisions multiple times.

The implantable device can be placed in the desired location within the eye according to surgical procedures, such as those known in the art. For example, the implantable device can be inserted through a sclerotomy into the suprachoroid. In such a procedure, the sclera is cut to expose the suprachoroid. The implantable device can then be positioned and inserted on either side of the incision. Alternatively, a partial-thickness scleral trap-door can be fashioned over the suprachoroid or an avascular region of the eye. The implantable device can then be inserted, and the scleral flap sewn back into place to secure the implant. Alternatively, the implantable device may be inserted so as to directly communicate with the vitreal chamber. To achieve this, a partial thickness scleral trap door flap is cut over an avascular region, such as the pars plana, to remove the eye coat. A hole (or holes) is made through the floor of the scleral bed to communicate with the base of the vitreous body through the pars plana. The implantable device is positioned over the hole within the scleral bed and the flap of the trap door is sewn back into place. Such placement of the implantable device will allow for the ready diffusion of the drug into the vitreous and into the intraocular structure.

If desired, the implantable device may be sealed within a package (e.g., sterile blister package) prior to use. The materials and manner in which the package is sealed may vary as is known in the art. In one embodiment, for instance, the package may contain a substrate that includes any number of layers desired to achieve the desired level of protective properties, such as 1 or more, in some embodiments from 1 to 4 layers, and in some embodiments, from 1 to 3 layers. Typically, the substrate contains a polymer film, such as those formed from a polyolefin (e.g., ethylene copolymers, propylene copolymers, propylene homopolymers, etc.), polyester (e.g., polyethylene terephthalate, polyethylene naphthalate, polybutylene terephthalate, etc.), vinyl chloride polymer, vinyl chloridine polymer, ionomer, etc., as well as combinations thereof. One or multiple panels of the film may be sealed together (e.g., heat sealed), such as at the peripheral edges, to form a cavity within which the device may be stored. For example, a single film may be folded at one or more points and sealed along its periphery to define the cavity within with the device is located. To use the device, the package may be opened, such as by breaking the seal, and the device may then be removed and implanted into a patient.

The present invention may be better understood by the following examples.

### Test Methods

Drug Release: The release of a pharmaceutical formulation (e.g., lysozyme) may be determined using an in vitro method. More particularly, implantable device samples may be placed in 150 milliliters of an aqueous PBS buffer solution. The solutions are enclosed in stoppered centrifuge tubes. The flasks are then placed into a temperature-controlled incubator and continuously shaken at 100 rpm. A temperature of 37° C. is maintained through the release experiments to mimic in vivo conditions. Samples are taken in regular time intervals by completely exchanging the aqueous buffer solution. The concentration of an antibody in solution may be determined via UV/Vis absorption spectroscopy using a Cary 3500 split beam instrument. From this data, the amount of the antibody released per sampling interval (microgram per day) may be calculated and plotted over time (day). Further, the cumulative release ratio of the antibody may be calculated as a percentage by dividing the amount of the antibody released at each sampling interval by the total amount of antibody initially present, and then multiplying this number by 100. This percentage is then plotted over time (day).

### Examples 1-3

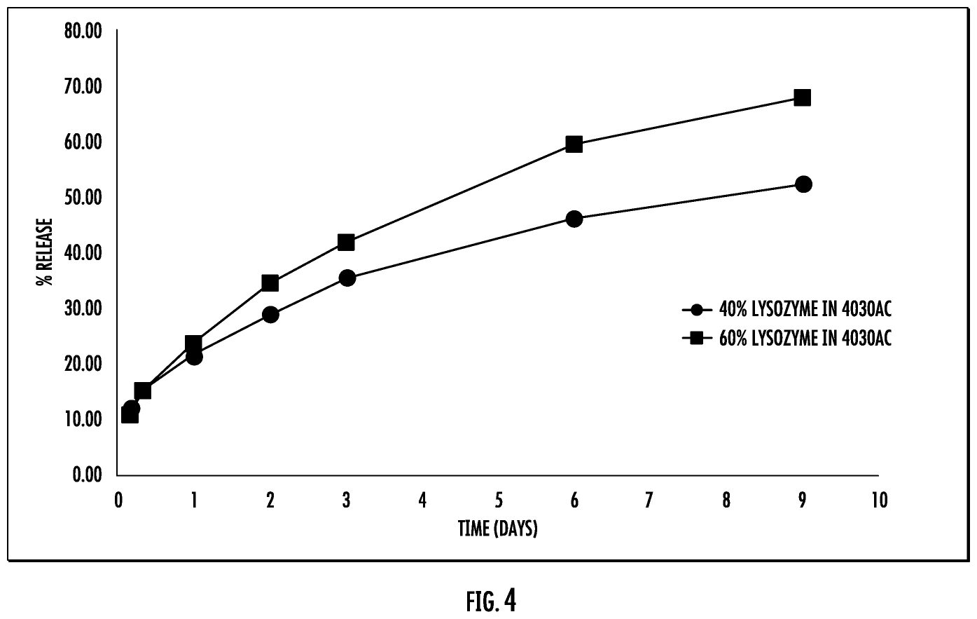

A rod-shaped monolithic implant containing human plasma derived IgG antibody was produced via extrusion. For Example 1, the device contained 60 wt. % Ateva® 4030AC and 40 wt. % IgG antibody. For Example 2, the device contained 50 wt. % Ateva® 4030AC and 50 wt. % IgG antibody. For Example 3, the device contained 55 wt. % Ateva® 4030AC and 45 wt. % IgG antibody. The devices of Examples 1-3 were formed by melt extruding the components using a 11 mm twin-screw extruder. Extrusion was accomplished using a screw speed of 50 rpm with barrel temperatures set to achieve a nominal melt temperature of 60° C. The rods had a diameter of 3.5 mm and were cut to a length of 3 cm for elution testing. The release of IgG from the rods was measured in PBS buffer in a shaking incubator maintained at 37° C. At regular intervals, the buffer was exchanged with fresh buffer, and the removed buffer characterized using UV-Vis absorbance spectroscopy to measure the concentration of IgG antibody released. The resulting cumulative release rate (%) over 160 days is shown in FIG. 1 and the elution data for Example 1 as expressed in total mass per unit surface area of the sample is shown in FIG. 4. For Example 1, only about 30% of the total antibody was eluted in 160 days. For Example 2, almost 60% of the total antibody was released in less than 20 days. For Example 3, about 25% of the antibody was eluted around day 21. Results for Example 1 are shown in Table 1 below. Results for Example 2 are shown in Table 2 below. Results for Example 3 are shown in Table 3 below

TABLE 1
Example 1

| | Area normalized | Total % |
|---|---|---|
| Time (Days) | release (mg/cm²) | released |
| 1 | 0.25 | 1.37 |
| 2 | 0.37 | 2.04 |
| 6 | 1.30 | 7.09 |
| 21 | 2.04 | 11.13 |
| 38 | 2.34 | 12.76 |
| 86 | 3.00 | 16.37 |
| 160 | 5.16 | 28.13 |

TABLE 2
Example 2

| | Area normalized | Total % |
|---|---|---|
| Time (Days) | release (mg/cm²) | released |
| 1 | 5.77 | 26.67 |
| 2 | 7.27 | 33.52 |
| 3 | 8.28 | 38.15 |
| 6 | 10.20 | 46.94 |
| 13 | 12.38 | 56.87 |

TABLE 3
Example 3

| | Area normalized | |
|---|---|---|
| Time (Days) | release (mg/cm²) | Total % released |
| 1 | 1.81 | 7.07 |
| 2 | 2.50 | 9.78 |
| 3 | 3.03 | 11.87 |
| 7 | 4.37 | 17.12 |
| 14 | 5.76 | 22.54 |
| 21 | 6.65 | 25.94 |

### Example 4

A pharmaceutical formulation was initially formed from the following components in Table 4:

TABLE 4

| Component | Concentration (wt. %) |
|---|---|
| Trastuzumab biosimilar (Syd Labs) | 51.3 |
| Trehalose | 46.6 |
| L-Histidine monohydrochloride | 1.2 |
| monohydrate | |
| L-Histidine | 0.7 |
| Sorbitan, monododecanoate | 0.2 |

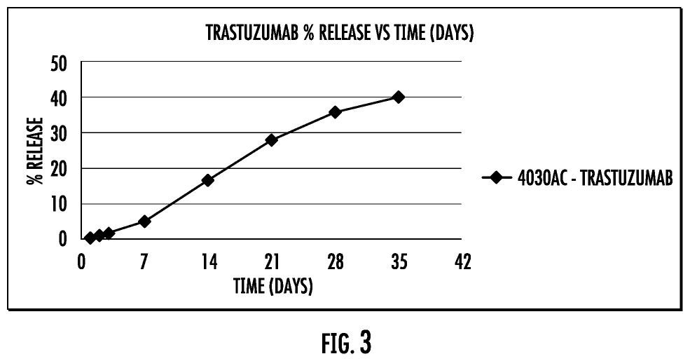

To form the formulation, the trastuzumab biosimilar was first lyophilized from an aqueous buffer and then the additional excipients were combined with the antibody to form a solid powder. Once formed, the powder was incorporated into an implantable device so that the resulting device contained 55 wt. % Ateva® 4030AC and 45 wt. % of the powder. The device was formed by melt extruding the components using a 11 mm twin-screw extruder. Extrusion was accomplished using a screw speed of 55 rpm with barrel temperatures set to achieve a nominal melt temperature of 60° C. The rod had a diameter of 3 mm and was cut to a length of 1 cm for elution testing. The release of the trastuzumab biosimilar from the rod was measured in PBS buffer in a shaking incubator maintained at 37° C. At regular intervals, the buffer was exchanged with fresh buffer, and the removed buffer characterized using UV-Vis absorbance spectroscopy to measure the concentration of trastuzumab antibody released. The resulting cumulative release rate (%) over 35 days is shown in FIG. 3. Approximately 40% of the total antibody was released in 35 days.

Results for Example 4 are shown in Table 5 below.

TABLE 5
Example 4

| | Area normalized | |
|---|---|---|
| Time (Days) | release (mg/cm²) | Total % released |
| 1 | 0.310 | 0.340 |
| 2 | 0.440 | 1.123 |
| 3 | 0.544 | 1.751 |
| 7 | 1.13 | 5.29 |
| 14 | 3.02 | 16.77 |
| 21 | 4.87 | 28.00 |
| 28 | 6.15 | 35.78 |
| 35 | 6.86 | 40.16 |
| 42 | 7.27 | 42.64 |
| 49 | 7.57 | 44.44 |
| 56 | 7.81 | 45.95 |
| 63 | 7.95 | 46.80 |
| 77 | 8.07 | 47.53 |
| 84 | 8.18 | 48.18 |
| 91 | 8.29 | 48.84 |
| 98 | 8.42 | 49.65 |
| 105 | 8.53 | 50.35 |
| 112 | 8.63 | 50.96 |
| 119 | 8.74 | 51.67 |
| 126 | 8.87 | 52.47 |
| 140 | 9.10 | 53.86 |
| 154 | 9.30 | 55.11 |
| 182 | 9.58 | 56.81 |

### Examples 5-6

A rod-shaped monolithic implant containing lysozyme was produced via extrusion. For Example 5, the device contained 60 wt. % Ateva® 4030AC and 40 wt. % lysozyme. For Example 6, the device contained 45 wt. % Ateva® 4030AC and 60 wt. % lysozyme. The devices of both examples were formed by melt extruding the components using a 11 mm twin-screw extruder. Extrusion was accomplished using a screw speed of 100 rpm with barrel temperatures set to achieve a nominal melt temperature of 75° C. The rods had a diameter of 3.5 mm and were cut to a length of 3 cm for elution testing. The release of lysozyme from the rods was measured in PBS buffer in a shaking incubator maintained at 37° C. At regular intervals, the buffer was exchanged with fresh buffer, and the removed buffer characterized using UV-Vis absorbance spectroscopy to measure the concentration of lysozyme released. The resulting cumulative release rate (%) over 9 days is shown in FIG. 4. For Example 5, about 50% of the total lysozyme was eluted in 9 days. For Example 6, greater than 60% of the total lysozyme was released in less than 9 days. Results for Examples 5-6 are shown in Tables 6 and 7 below.

TABLE 6
Example 5

| | Area normalized | Total % |
|---|---|---|
| Time | release (mg/cm²) | released |
| 4 hours | 6.11 | 11.98 |
| 8 hours | 7.86 | 15.40 |
| 24 hours | 11.17 | 21.89 |
| 48 hours | 14.79 | 29.00 |
| 72 hours | 18.09 | 35.45 |
| 6 days | 23.46 | 45.99 |
| 9 days | 26.63 | 52.21 |

TABLE 7
Example 6

| | Area normalized | Total % |
|---|---|---|
| Time (Days) | release (mg/cm²) | released |
| 4 hours | 3.52 | 10.87 |
| 8 hours | 4.96 | 15.32 |
| 24 hours | 7.70 | 23.81 |
| 48 hours | 11.17 | 34.54 |
| 72 hours | 13.47 | 41.65 |
| 6 days | 19.21 | 59.39 |
| 9 days | 21.90 | 67.69 |

### Examples 7-11

To characterize the protein after drug release, freshly prepared solutions of human-plasma derived IgG antibody as well as solutions collected at selected time points during the elution study described in Example 1 were characterized via size exclusion chromatography (SEC) using an HPLC system equipped with an Agilent AdvanceBio SEC column. Phosphate buffer (0.15 M; pH 7.0) was used as elution media and flow rate was maintained at 1 mL/min. The detection wavelength was set at 280 nm. These samples are described in the below table.

| Example | Description |
|---|---|
| Ex. 7 | Freshly prepared IgG solution (1 mg/ml) |
| Ex. 8 | Solution collected at 6-day time point from Ex. 1 |
| Ex. 9 | Solution collected at 21-day time point from Ex. 1 |
| Ex. 10 | Solution collected at 86-day time point from Ex. 1 |
| Ex. 11 | Solution collected at 160-day |
| | time point from Ex. 1 |

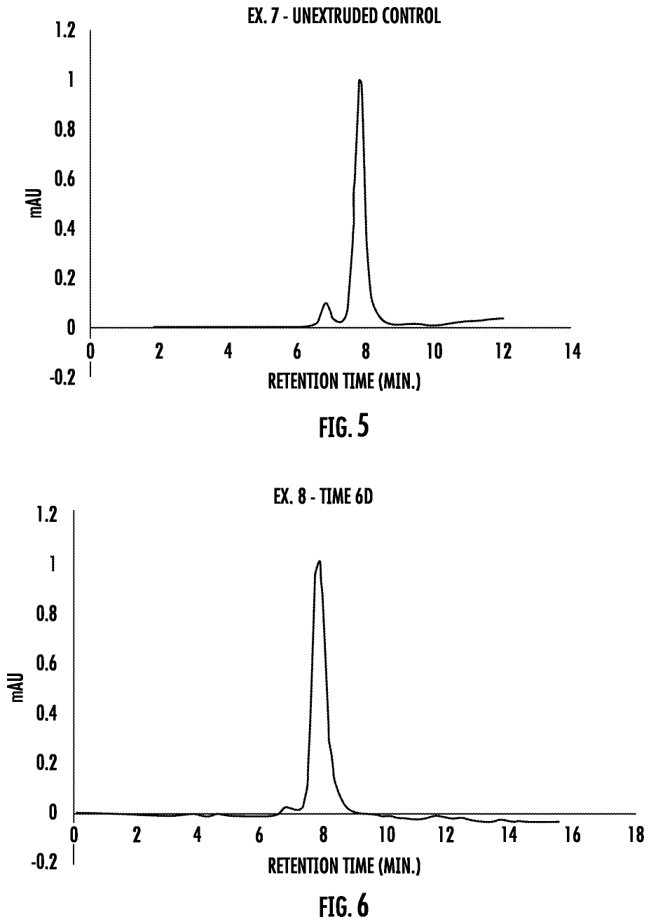

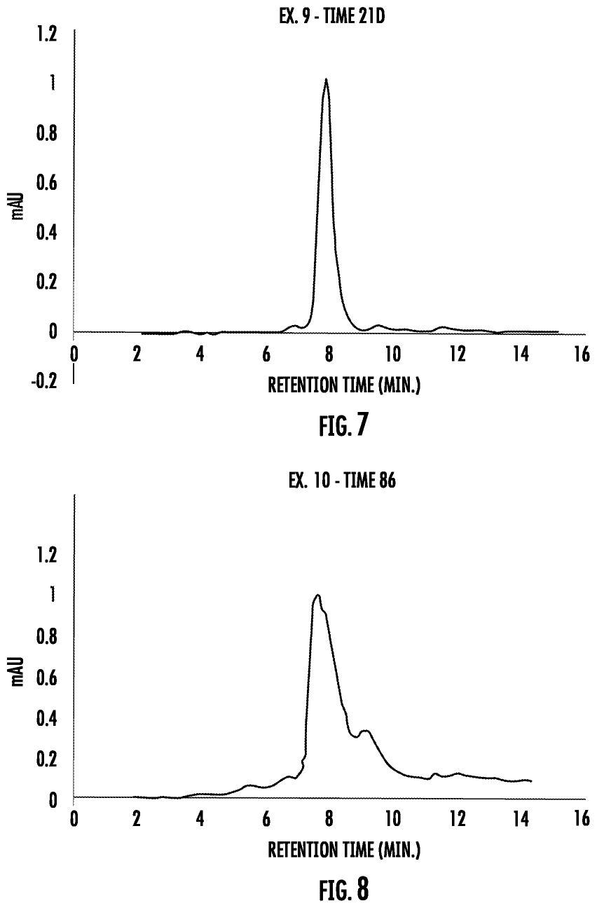

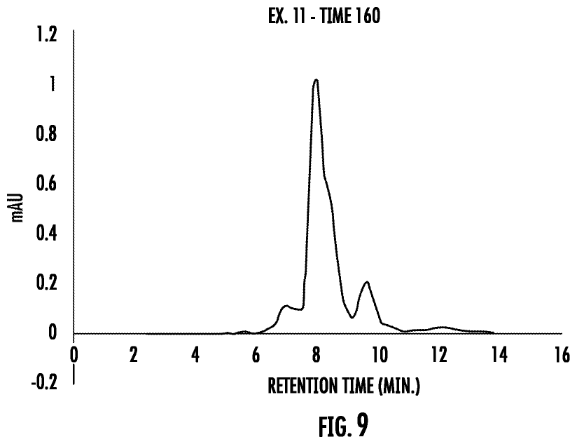

The SEC chromatograms are plotted in FIGS. 5-9. Freshly prepared solution (Ex. 7) exhibits a large protein monomer peak (8-minute retention time) as well as a small protein dimer peak (7-minute retention time) (see FIG. 5). At the 6 and 21-day time points (Ex. 8 and Ex. 9) the collected solutions show well defined monomer peaks and small dimer peaks (see FIGS. 6-7). At the 86-day (Ex. 10) and 160-day (Ex. 11) time points significant protein degradation becomes apparent via the introduction of a new peak present at higher retention times (9-minute retention time). Additionally, some protein aggregation is detected in Ex. 10 through the introduction of a small peak at lower retention times (5.5 minutes). (See FIGS. 8-9).

These and other modifications and variations of the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed is:

1. A monolithic implantable device for delivery of an antibody, the implantable device comprising a polymer matrix within which is dispersed a pharmaceutical formulation that includes one or more therapeutic agents and optionally, one or more excipients, wherein the therapeutic agents contain the antibody and the polymer matrix contains a hydrophobic polymer, wherein within a time period of 35 days, the device exhibits a cumulative weight-based release ratio of the antibody of from about 20% to about 60%.

2. The monolithic implantable device of claim 1, wherein the weight ratio of the pharmaceutical formulation to the polymer matrix is from about 0.7 to about 1.

3. The monolithic implantable device of claim 2, wherein the weight ratio of the one or more therapeutic agents to the polymer matrix is from about 0.7 to about 1.

4. The monolithic implantable device of claim 2, wherein the weight ratio of the one or more therapeutic agents to the polymer matrix is from about 0.3 to about 1.

5. The monolithic implantable device of claim 1, wherein the pharmaceutical formulation constitutes from about 30 wt. % to about 50 wt. % of the device and the polymer matrix constitutes from about 50 wt. % to about 70 wt. % of the device.

6. The monolithic implantable device of claim 1, wherein within a time period of 15 days, the device exhibits a cumulative weight-based release ratio of the antibody of from about 10% to about 55%.

7. The monolithic implantable device of claim 1, wherein the hydrophobic polymer is a copolymer that contains a polar monomer and an olefin monomer.

8. The monolithic implantable device of claim 7, wherein the polar monomer content is from 10 wt. % to about 60 wt. % of the copolymer.

9. The monolithic implantable device of claim 1, wherein the hydrophobic polymer has a melt flow index of from about 0.2 to about 100 grams per 10 minutes as determined in accordance with ASTM D1238-20 at a temperature of 190° C. and a load of 2.16 kilograms and/or a melting temperature of from about 20° C. to about 70° C. as determined in accordance with ASTM D3418-21.

10. The monolithic implantable device of claim 1, wherein the hydrophobic polymer includes an ethylene vinyl acetate copolymer.

11. The monolithic implantable device of claim 1, wherein hydrophobic polymers constitute the entire polymer matrix.

12. The monolithic implantable device of claim 1, wherein the antibody includes a monoclonal antibody.

13. The monolithic implantable device of claim 12, wherein the monoclonal antibody includes an anti-PD-1 antibody, anti-PD-L1 antibody, anti-HER2 antibody, anti-VEGF antibody, or a combination thereof.

14. The monolithic implantable device of claim 13, wherein the anti-HER2 antibody includes trastuzumab, pertuzumab, margetuximab, or an antigen-binding variant thereof.

15. The monolithic implantable device of claim 13, wherein the anti-VEGF antibody includes bevacizumab, ranibizumab, or an antigen-binding variant thereof.

16. The monolithic implantable device of claim 1, wherein the pharmaceutical formulation includes one or more excipients.

17. The monolithic implantable device of claim 16, wherein the excipients include a buffering agent.

18. The monolithic implantable device of claim 17, wherein the buffering agent includes a histidine buffer.

19. The monolithic implantable device of claim 16, wherein the excipients include a saccharide.

20. The monolithic implantable device of claim 19, wherein the saccharide includes dextrose, fructose, galactose, ribose, deoxyribose, sucrose, lactose, maltose, trehalose, xylitol, sorbitol, mannitol, maltitol, erythritol, galactitol, inositol, lactitol, or a combination thereof.

21. The monolithic implantable device of claim 16, wherein the excipients include a surfactant.

22. The monolithic implantable device of claim 21, wherein the surfactant includes a sorbitan fatty acid ester modified with a polyoxyethylene.

23. The monolithic implantable device of claim 1, wherein the formulation is the form of a dehydrated particulate material.

24. The monolithic implantable device claim 1, wherein the device has a generally circular cross-sectional shape.

25. The monolithic implantable device of claim 1, wherein the device is in the form of a cylinder.

26. The monolithic implantable device of claim 1, wherein the device has a thickness of from about 0.1 to about 10 millimeters and/or a length of from about 1 to about 250 millimeters.
